(19) United States
(12) Patent Application Publication
(10) Pub. No.: US 2009/0221850 A1
(43) Pub. Date: Sep. 3, 2009

(54) METHOD FOR PREPARING ALKOXYAMINES BY PHOTOLYSIS OF DITHIOCARBAMATES

(76) Inventors: Denis Bertin, Marseille (FR); Jean-Luc Couturier, Lyon (FR); Didier Gigmes, Marseille (FR); Olivier Guerret, La Tour De Salvagny (FR); Yohann Guillaneuf, Marseille (FR)

Correspondence Address: ARKEMA INC.;PATENT DEPARTMENT - 26TH FLOOR, 2000 MARKET STREET, PHILADELPHIA, PA, 19103-3222, US

(21) Appl. No.: 11/911,862

(22) PCT Filed: Apr. 13, 2006
(86) PCT No.: PCT/FR06/00808

§ 371 Date: Jun. 3, 2008

(30) Foreign Application Priority Data
Apr. 19, 2005 (FR) 0503878

(51) Int. Cl. C07C 205/02

(52) U.S. Cl. 562/553

(57) ABSTRACT

The present invention relates to a novel process for preparing alkoxyamines from a nitroxide and a dithiocarbamate by photolysis reaction.

This process, which does not generate effluent containing metals, may be carried out in batch or in continuous mode and takes place at a lower temperature than the known processes for synthesizing alkoxyamines.

### TECHNICAL FIELD

The invention relates to a process for preparing alkoxyamines by photochemical reaction between a dithiocarbamate-type organic compound and a nitroxide. This process is particularly suitable for the synthesis of the N-(tert-butyl)-N-(1-diethylphosphono-2,2-dimethylpropyl)-O-(2-carboxylprop-2-yl)hydroxylamine alkoxy-amine.

The alkoxyamines prepared according to the process of the invention may be used for synthesizing polymer materials or polymer additives (for example of the type: dispersants, rheology modifiers, emulsifiers, impact additives) in the fields of plastics, elastomers, paints, adhesives, cosmetics, paper, hydraulic binders (cement or plaster), ceramics, bitumens, lubricants, oil production, emulsification or encapsulation (controlled salting-out of active principles).

### PRIOR ART

The synthesis of alkoxyamines may be carried out by various methods. Mention may be made, for example, of the revue by A. Studer in Chem. Soc. Rev., 2004, 33, 267-273, which recapitulates the existing synthesis processes. The most commonly used method is trapping of a radical by a nitroxide; the radical is generally generated from an activated halogenated compound and from a copper complex, or by the thermal reaction of a peroxide on a compound having an activated C—H bond, or else by photolysis of an azo compound as described in U.S. Pat. No. 4,581,429.

These processes require difficult purification steps, which may result in significant amounts of effluents that may or may not contain metals, or steps that cannot be scaled-up industrially such as chromatography on silica.

The photolysis of dithiocarbamate-type compounds is known. Mention may be made, for example, of the publication by T. Otsu et al. in Makromol. Chem., Rapid Commun. 1982, 3, 133-140 where this reaction was used to initiate photopolymerizations but it was never used in the presence of nitroxides to synthesize alkoxyamines.

### SUMMARY OF THE INVENTION

The invention relates to an industrial process for preparing alkoxyamines by photolysis of dithiocarbamate-type organic compounds that makes it possible to solve the drawbacks mentioned above.

More specifically, the invention relates to a process for preparing alkoxyamines R₁ONR₂R₃ by reaction of dithiocarbamate-type organic compounds with nitroxide-type compounds according to the scheme:

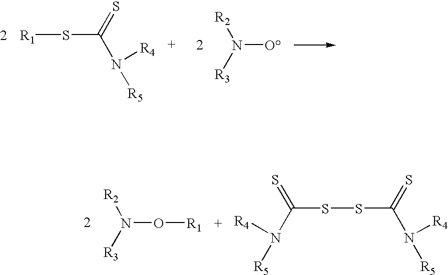

where R₁ is an aromatic or non-aromatic, cyclic or non-cyclic, linear or branched, hydrocarbon-based radical and which may contain hetroatoms, in particular O and N, or alkali metals, in particular Na and K;

R₂ and R₃ are aromatic or non-aromatic, cyclic or non-cyclic, linear or branched, hydrocarbon-based radicals, which may be identical or different, having from 1 to 30 carbon atoms which may contain hetroatoms, in particular O, N, S, P and Si, R₂ and R₃ possibly being connected to form cyclic structures with the nitrogen atom; and

R₄ and R₅ are aromatic or non-aromatic, cyclic or non-cyclic, linear or branched, hydrocarbon-based radicals, which may be identical or different, having from 1 to 18 carbon atoms, R₄ and R₅ possibly optionally forming cyclic structures with the nitrogen atom.

The process according to the invention is particularly suitable for the synthesis of heat-sensitive alkoxyamines derived from the nitroxide N-tert-butyl-1-diethylphosphono-2,2-dimethylpropyl nitroxide described in EP 1 349 862.

It is also particularly suitable for the synthesis of the alkoxyamine N-(tert-butyl)-N-(1-diethylphosphono-2,2-dimethylpropyl)-O-(2-carboxylprop-2-yl)hydroxylamine of formula (I) described in WO 2004/014926:

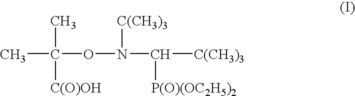

Finally, the process can also be applied for converting a dithiocarbamate function present in a polymer to an alkoxyamine function.

The process according to the invention consists in the photolysis of the dithiocarbamate-type compound in the presence of the nitroxide.

The organic compounds of dithiocarbamate type may be synthesized in several ways. Mention may be made, for example, of the nucleophilic substitution between a bromo derivative and a sodium dialkyldithiocarbamate as described by G. Nachmias in Annales de chimie, 1952, volume 7, p. 584-631, the haloform synthesis (chloroform+acetone+sodium dialkyldithiocarbamate) as described in US 2003/0120101 or else the reaction between an azo compound and a dialkylthiuram disulphide as described by Zard et al. in Tetrahedron Letters, 1999, 40, 277-280.

The dithiocarbamate/nitroxide molar ratio is preferably between 1 and 2. The photolysis may, for example, be carried out using one or more mercury vapour lamps emitting radiation having a wavelength between 200 and 600 nm. The reaction is generally carried out at a temperature between 0 and 60° C. in the presence of a solvent. The solvent or solvents are chosen as a function of the substrates used; among the solvents which are generally used, mention may be made of water, alcohol such as methanol, ethanol, propanol, isopropanol, tert-butanol or ethylene glycol, ethers such as THF, esters such as ethyl acetate, nitriles such as acetonitrile, aromatics such as benzene, toluene, ethylbenzene or tert-butylbenzene, alkanes such as cyclohexane, or chlorinated solvents such as carbon tetrachloride or a mixture of several of the aforementioned solvents. The photolysis reaction generally takes place under an inert atmosphere (nitrogen, argon) and with vigorous stirring, either by nitrogen sparging via a diffuser or by means of a recirculating pump.

The alkoxyamine obtained is then isolated or purified according to the methods known to a person skilled in the art.

The by-products of a thiuram disulphide type may be separated from the alkoxyamine by various methods, for example by precipitation or by selective liquid extraction. Optionally, the by-product of thiuram disulphide type may be reconverted to dithiocarbamate by a thermal reaction with an azo compound according to the method described, for example, by Zard et al. in Tetrahedron Letters, 1999, 40, 277-280, of which the scheme is explained in detail below:

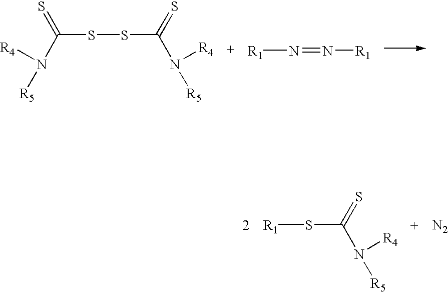

The process of the present invention may be carried out in batch mode or in continuous mode. When proceeding under dithiocarbamate/nitroxide conditions close to the stoichiometry, the alkoxyamine yields of the process according to the invention are particularly high.

Moreover, the process has the additional advantage of functioning at low temperature, which makes it possible to synthesize, without difficulty and without any particular precaution, heat-sensitive alkoxyamines, such as for example those derived from the nitroxide N-tert-butyl-1-diethylphosphono-2,2-dimethylpropyl nitroxide and even more particularly for the synthesis of the alkoxyamine N-(tert-butyl)-N-(1-diethylphosphono-2,2-dimethylpropyl)-O-(2-carboxylprop-2-yl)hydroxylamine.

Furthermore, once the photolysis reaction has been completed, purification of the alkoxyamine is particularly easy and does not generate any effluent containing metals, which is an undeniable advantage from an environmental point of view.

The alkoxyamines prepared according to the process of the present invention may be used as initiators for a radical reaction of the ring-closure type or as radical polymerization initiators, in particular for the technique of controlled radical polymerization.

Thus, another subject of the invention is a process for preparing a polymer material comprising at least one step of polymerizing a polymerizable monomer by a radical route, this polymerization step being carried out in the presence of an alkoxyamine obtained as described previously.

### EXAMPLES

### Example 1

### Preparation of S-(1-methyl-1-carboxyethyl)-N,N-diethyldithiocarbamate

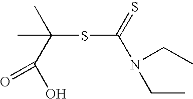

Introduced into a 100 ml glass round-bottomed flask were 6.68 g of 2-bromoisobutyric acid (0.04 mol) and 60 ml of water. The medium was neutralized by addition of Na₂CO₃ up to pH=9. Next, 9 g of sodium diethyldithiocarbamate trihydrate (0.04 mol) were added, then the medium was left stirring for 15 hours at ambient temperature. The reaction mixture was then acidified to pH=2 by addition of 33% hydrochloric acid. The white precipitate which appeared was recovered by filtration, then dried under vacuum. Thus, 3.1 g of S-(1-methyl-1-carboxyethyl)-N,N-diethyldithiocarbamate was obtained (yield=33%) which was characterized by ¹H NMR. ¹H NMR (CDCl₃-300 MHz): 1.25-1.30 ppm (m, 6H); 1.78 ppm (s, 6H); 3.5-4 ppm (m, 4H). Furthermore, N-tert-butyl-1-diethylphosphono-2,2-dimethylpropyl nitroxide (SG1) was prepared according to the teaching of EP 1 349 862.

Introduced into the photochemical reactor were: 4.8 g of dithiocarbamate prepared previously (20.4 mmol; 1.3 equivalents), 4.62 g of SG1 (15.7 mmol; 1 equivalent) and 350 ml of ethanol. The reactor was degassed with nitrogen, then the lamp was lit and the reaction was carried out for 4 hours at 20° C. until decolouration of the reaction mixture. The photochemical reactor used was a 350 ml glass reactor equipped with a holder for a quartz jacketed lamp. The lamp used was a medium pressure mercury vapour lamp having a power of 150 W and emitting between 254 nm and 350 mn (reference TQ150 from Heraeus).

The reaction mixture was then poured into 200 ml of water containing 2 ml of 33% hydrochloric acid. The precipitate of pale yellow colour which appeared was filtered. The powder was taken up by 200 ml of water containing sodium carbonate at pH=9. The tetraethylthiuram disulphide which remained in suspension in the form of a yellow solid was removed by filtration. The filtrate was acidified to pH=2 by addition of 33% HCl. The white precipitate formed was filtered, washed with water, then dried under vacuum. 4.5 g of alkoxyamine (I) was obtained (yield=75%).

The product was characterized by ¹H, ¹³C and ³¹P NMR. The results were in agreement with those published in WO 2004/014926.

Microanalysis was also carried out on the product formed, the results of which are combined in the table below:

| | Element | | |
|---|---|---|---|
| | C | H | N |
| Theoretical % | 53.53 | 9.51 | 3.67 |
| Experimental % | 53.78 | 9.57 | 3.69 |

### Example 2

The procedure from Example 1 was followed with the exception of the dithiocarbamate/nitroxide ratio which was brought to 1.5 instead of 1.3, i.e. 6.93 g of dithiocarbamate (23.5 mmol) and 4.62 g of SG1 (15.7 mmol)

Thus, 5.4 g of alkoxyamine (I) was recovered, i.e. a yield of 90%.

1. Process for preparing alkoxyamines of formula R₁ONR₂R₃ by photolysis reaction of a dithiocarbamate with a nitroxide according to the scheme: 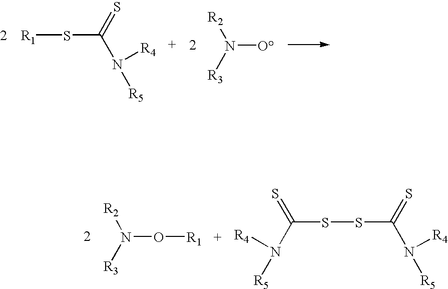
where R₁ is an aromatic, non-aromatic, cyclic, non-cyclic, linear or branched, hydrocarbon-based radical and which may contain first hetroatoms, or alkali metals;
R₂ and R₃ are aromatic, non-aromatic, cyclic, non-cyclic, linear or branched, hydrocarbon-based radicals, which may be identical or different, having from 1 to 30 carbon atoms which may contain second hetroatoms R₂ and R₃ possibly being connected to form cyclic structures with the nitrogen atom; and
R₄ and R₅ are aromatic, non-aromatic, cyclic, non-cyclic, linear or branched, hydrocarbon-based radicals, which may be identical or different, having from 1 to 18 carbon atoms, R₄ and R₅ possibly optionally forming cyclic structures with the nitrogen atom, followed by a step of purifying the alkoxyamine obtained.

2. Process according to claim 1, in accordance with a photolysis reaction in a wavelength range lying between 200 and 600 nm.

3. Process according to claim 1, at a temperature between 0 and 60° C. in the presence of a solvent, chosen from water, alcohol, ethers, esters, nitrites, aromatics, alkanes, or chlorinated solvents alone or as a mixture.

4. Process according to claim 1, under an inert atmosphere and with vigorous stirring.

5. Process according to claim 1, characterized in that said nitroxide N-tert-butyl-1-diethylphosphono-2,2-dimethylpropyl nitroxide.

6. Process according to claim 5, characterized in that the N-(tert-butyl)-N-(1-diethylphosphono-2,2-dimethylpropyl)-O-(2-carboxylprop-2-yl)hydroxylamine alkoxyamine is prepared from N-tert-butyl-1-diethylphosphono-2,2-dimethylpropyl nitroxide and S-(1-methyl-1-carboxyethyl)-N,N-diethyldithiocarbamate or S-(1-methyl-1-carboxyethyl)-N,N-dimethyldithiocarbamate.

7. Process according to claim 1, characterized in that said first hetroatoms are selected from O or N.

8. Process according to claim 1, characterized in that said alkali metal is selected from Na or K.

9. Process according to claim 1, characterized in that said second hetroatoms are selected from O, N, S, P or Si.

10. Process according to claim 3, characterized in that said alcohol is selected from methanol, ethanol, propanol, isopropanol, tert-butanol or ethylene glycol

11. Process according to claim 3, characterized in that said ether is THF.

12. Process according to claim 3, characterized in that said ester is ethyl acetate

13. Process according to claim 3, characterized in that said nitrile is acetonitrile.

14. Process according to claim 3, characterized in that said aromatic is selected from benzene, toluene, ethylbenzene or tert-butylbenzene.

15. Process according to claim 3, characterized in that said alkane is cyclohexane.

16. Process according to claim 3, characterized in that said chlorinated solvent is carbon tetrachloride.

17. Process according to claim 4, characterized in that said inert atmosphere is nitrogen or argon.
